(19) United States
(12) Patent Application Publication
(10) Pub. No.: US 2026/0287579 A1
(43) Pub. Date: Sep. 24, 2026

(54) METHOD FOR NON-INVASIVE HYPOXIA DETECTION

(71) Applicants: BioVentures, LLC, Little Rock, AR (US); Board of Trustees of the University of Arkansas, Little Rock, AR (US)

(72) Inventors: Robert J. Griffin, Little Rock, AR (US); Azemat Jamshidi-Parsian, Little Rock, AR (US); Samir V. Jenkins, Little Rock, AR (US); Ruud PM Dings, Little Rock, AR (US); Narasimhan Rajaram, Bentonville, AR (US); Varsha Karunakaran, Fayetteville, AR (US); Rajshekhar Kore, Sherwood, AR (US)

(21) Appl. No.: 19/474,520

(22) PCT Filed: Apr. 15, 2024
(86) PCT No.: PCT/US2024/024593

§ 371 Date: Oct. 10, 2025

Related U.S. Application Data
(60) Provisional application No. 63/459,070, filed on Apr. 13, 2023.
(60) Provisional application No. 63/459,779, filed on Apr. 17, 2023.

(51) Int. Cl. G01N 33/53; G01N 21/64; G01N 21/65

(52) U.S. Cl. CPC G01N 33/5308; G01N 21/6428; G01N 21/65; G01N 2800/7038

(57) ABSTRACT

A method for detecting hypoxia in a patient is disclosed. The method includes administering to the patient an effective amount of a nitroimidazole, drawing blood from the patient and separating the serum of the blood, isolating an extracellular vesicle fraction from the serum, and conducting a test for detecting the nitroimidazole in the extracellular fraction. The test result from the test indicates the amount of hypoxic cells in said patient. The test result may be compared to a normal test result to determine the presence or absence of a medical condition.

### CROSS-REFERENCE TO RELATED APPLICATIONS

This application claims the benefit of U.S. Provisional Application No. 63/459,070, entitled “Method and Device for Non-Invasive Hypoxia Detection” and filed on Apr. 13, 2023, and U.S. Provisional Application No. 63/459,779, entitled “Method and Device for Non-Invasive Hypoxia Detection” and filed on Apr. 17, 2023. The complete disclosure of said provisional applications is hereby incorporated by reference.

### STATEMENT REGARDING FEDERALLY SPONSORED RESEARCH OR DEVELOPMENT

This invention was made with government support from grant no. 1R01CA238025 awarded by the National Institute of Health. The government has certain rights in the invention.

### BACKGROUND

Hypoxia or the lack of oxygen has been associated with poor long-term disease outcome in several cancer types. Early detection or persistent detection of hypoxia could signal the beginnings of a disease or the damage to tissue/organs. The gold standard for detection of hypoxic cells and tissue involves the injection of nitroimidazole-based hypoxia probes. These nitroimidazoles form stable adducts in the absence of oxygen (hypoxia). By excising tissue from animals and patients, the presence of hypoxic regions can be detected using immunohistochemical assays of these nitroimidazoles. However, this technique is invasive, highly subjective and time consuming. Recent advances in noninvasive early detection of diseases, such as cancer include the identification of circulating cancer cells, DNA and other vesicles shed by tumors into the blood vessels. However, these methods are not perfect because the mere detection of circulating tumor cells is not a perfect indicator of poor disease-free survival. Exosomes are one species of vesicle that are known to be secreted by cancer cells, and they are small membrane bound vesicles which function as cargo carrier entities that transport small molecules like nucleic acids, proteins, and lipids between cells. Exosomes and other vesicles secreted by nitroimidazole treated hypoxic cells are expected to contain the nitroimidazole adducts. The possible detection of such hypoxic exosomes presents an excellent opportunity for early detection of diseases as well as progression or regression of such diseases, such as cancer and tissue ischemia after surgery or an accident. The inventors have established, using a number of standard cell biology type assays, and Raman imaging that a detection signal for pimonidazole-labeled vesicles or other biological materials from blood samples or tissue culture medium can be obtained, and the detection signal correlates with the amount/presence of severe hypoxia in a growing tumor or cells cultured in a hypoxic environment.

Raman spectroscopy is a non-invasive optical technique that offers the ability to probe biomolecular changes and visualize complex molecular heterogeneity directly from cells and tissues. Raman spectroscopy relies on the inelastic scattering of light, arising from its interactions with the biological specimen, to quantify the unique vibrational modes of molecules in its native context. Specifically, Raman spectra encode information on the vibrational structure of compositional proteins (e.g. collagen and elastin), glycosaminoglycans, proteoglycans, lipids, and nucleic acids allowing for cell and tissue assessments with a high degree of biomolecular specificity. The inventors have demonstrated the ability of Raman spectroscopy to discriminate between tumors treated with different immune checkpoint inhibitors and between radiation-resistant and sensitive tumors. Early results indicate that exosomes labeled with nitroimidazole compounds by virtue of originating in cells growing in a hypoxic environment can be discriminated by Raman imaging.

The goal of this project was to develop a noninvasive sensing approach to detect hypoxic exosomes as an early-stage marker of disease. Establishing an accurate and bedside sensing approach that relies on the functional state of a tumor (hypoxia) could increase opportunities for earlier detection of disease and improve treatment strategies based on functional knowledge of the disease. The inventors have established that there are peaks in the Raman spectra that correspond to hypoxia in exosomes isolated from hypoxic tissue culture conditions and surmise that Raman spectroscopy can be used to noninvasively sense hypoxic exosomes in patient blood samples. Compared with expensive, destructive, and time-consuming techniques, such as mass spectrometry, Raman spectroscopy provides the ability to sense biomolecular signatures noninvasively and rapidly within cells. Nitroimidazole-based hypoxia markers, such as pimonidazole, are typically used in animal and human studies to label hypoxic tissue. Pimonidazole is reduced to form stable protein adducts in thiol rich molecules under hypoxic conditions. The preliminary work detailed below has shown that pimonidazole has a unique and specific Raman spectral signature in its natural state. Proof-of-concept experiments conducted in exosomes derived from normoxic and hypoxic breast cancer cells revealed consistent biomolecular differences between the normoxic and hypoxic exosome samples as well as normoxic and hypoxic Pimonidazole treated samples. Most recently, the inventors have established that analysis of the immunoblotting results of protein samples of a simple fluorescence-based assay for bound anti-pimonidazole antibody can resolve the difference between oxic-derived and hypoxic-derived exosomes. Therefore, these findings have motivated the inventors to advance the Raman optical tool to reveal the biomolecular signatures of hypoxia in labeled exosomes from blood samples and continue to solidify the diagnostic/prognostic potential of monitoring hypoxia in a patient using a simple blood sample and a variety of protein detection strategies using anti-pimonidazole antibodies.

### DISCLOSURE OF THE INVENTION

The present invention is directed to methods for detecting hypoxia in a patient. The method preferably includes administering to the patient an effective amount of a nitroimidazole, drawing blood from the patient and separating the serum of the blood, isolating an extracellular vesicle fraction from the serum, and conducting a test for detecting the nitroimidazole in the extracellular fraction. The test result from the test indicates the amount of hypoxic cells in the patient. The test result may be compared to a normal test result to determine the presence or absence of a medical condition. The nitroimidazole used may be pimonidazole, etanidazole, or misonidazole. The test used may be a Western blot, flow cytometry, a plate reader assay, or Raman spectroscopy.

Most solid tumors that grow in humans have portions of their volume that become extremely hypoxic due to rapid cellular growth and dysfunctional vasculature. Circulating vesicles that are shed from cells in the body contain proteins and genes that relate to their environment suggesting certain features could be used to detect circulating vesicles secreted from the tumor. The inventors hypothesized that one approach that may be feasible would be to administering a nitroimidazole or other bioreductive sensing compound systemically. Nitroimidazoles are compounds that are preferentially reduced and bind to biological molecules in severe hypoxia levels exclusive to the solid tumor microenvironment or ischemic tissue and thus may selectively label vesicles being shed from these regions. Therefore, a blood based sample could be taken, labeled vesicles isolated and hypoxia-specific adducts detected by antibodies or by Raman or photoacoustic imaging ex vivo. The initial studies indicate that vesicle-specific proteins isolated from mice or from cell culture medium that have been labeled by pimonidazole in the bioreductive environment that occurs under hypoxia can be detected by western blotting, flow cytometry, fluorescence-based plate reader approaches and Raman spectroscopy. Densitometric analysis of western blots for pimonidazole adducts show up to a 4-fold elevation in total antibody binding in exosome samples from animals with growing tumors as compared to animals with no cancer present. There was also a 4-fold enhancement in percent of vesicles labeled in hypoxic conditions vs oxic conditions using flow cytometry and an approximate 2.5-fold increase in fluorescence signal when exosomes isolated from hypoxic cells were compared to those isolated from cells grown in normal oxygen. Unique Raman peaks are also present when comparing between control cells and vesicles harvested from hypoxic but not oxic culture conditions. Pimonidazole generated a strong Raman signal with distinct peaks. Further, isolated vesicles from 4T1 murine breast cancer cells labeled with pimonidazole in normoxic or hypoxic conditions showed key differences in spectral features. Using Principal Component Analysis, the inventors were able to clearly differentiate between normoxic and hypoxic vesicles. Overall, the ability to detect the presence of vesicles being shed from a hypoxic niche could lead to a non-invasive blood test for tumor presence, as well as a surrogate marker for treatment success or recurrence or a measure of other tissue ischemia in the body.

### BRIEF DESCRIPTION OF THE DRAWINGS

FIG. 1 is a Raman spectral pattern of Pimonidazole, a nitroimidazole-based hypoxia probe.

FIG. 2A is a Raman scattering spectra from exosomes derived from pimonidazole treated 4T1 murine breast cancer cells exposed to normoxic or hypoxic conditions. Normoxic exosomes with PIMO is the bottom line. Hypoxic exosomes with PIMO is top horizontal line showing increase in peak intensity.

FIG. 2B is Principal Component Analysis (PCA) classification of the Normoxia Pimonidazole and Hypoxia Pimonidazole differences. Hypoxic PIMO exosomes fall in the right upper and lower quadrants, while normoxic PIMO exosomes fall entirely in the left upper and lower quadrants.

FIG. 3 is the general chemical reaction that favors reduction of pimonidazole and binding to macromolecules in the cell.

FIG. 4A is a chemical reaction showing the bioreductive process by which pimonidazole can form covalent bonds with cellular macromolecules.

FIG. 4B is an immunohistochemistry based image showing the distribution of pimonidazole in a hypoxic tissue as detected with a fluorescently tagged anti-pimonidazole antibody. Animals were injected with pimonidazole i.p. (intraperitoneal injection) and 2 hours later tissue was harvested and immediately flash frozen. After cutting 3-5 um thick sections, the anti-pimonidazole antibody and blocking agents were added for 2 hours, washed away with three rinses of PBS, and the fluorescent signal from bound antibody was captured using a standard fluorescent microscope workstation with image capture software. Green is the fluorescence from the anti-pimonidazole antibody indicating where hypoxic cells are located in the tissue section and blue is the general nuclei staining to mark all cells.

FIG. 5A is 4T1 tumor tissue section that is stained for tumor vessels (CD31; red) and cellular nuclei (blue).

FIG. 5B is a 4T1 tumor tissue section that is stained for tumor hypoxia (pimonidazole; green) and cellular nuclei (blue).

FIG. 5C is an image of both signals co-localized (white) in 4T1 tumor tissue in relation to microvasculature in 4T1 tumor tissue as detected by immunohistochemistry

FIG. 5D is a graph showing the quantification of overall tumor vessels from the digital images captured by a fluorescent microscope in three different breast tumor types from mouse (4T1, SCK and MMTV).

FIG. 5E is a graph showing the quantification of hypoxia (total pimonidazole signal).

FIG. 5F is a graph showing the quantification of hypoxic tumor vessels (total colocalized signals).

FIG. 5G is an image showing the immunofluorescence analysis of vasculature (CD31; red) and hypoxia (pimonidazole; green) in non-diseased kidney from a Balb/C mouse.

FIG. 5H is an image showing the immunofluorescence analysis of vasculature (CD31; red) and hypoxia (pimonidazole; green) in spleen

FIG. 5I is an image showing the immunofluorescence analysis of vasculature (CD31; red) and hypoxia (pimonidazole; green) in liver indicates a lack of global and vessel hypoxia in normal tissue.

FIG. 6A is a schematic showing unlabeled microbubbles (MB) (left), pimonidazole-targeting MB (MBα-pimo) (middle), and MBα-pimo without pimonidazole present in the circulation (right).

FIG. 6B are brightfield microscopy images showing that MBα-pimo binds hypoxic 2H11 endothelial cells only in the presence of pimonidazole. MBα-pimo does not bind endothelial cells (shown in left image), unless pimonidazole is added (shown in middle image). MBα-pimo binds to the cell surface of hypoxic endothelial cells (shown in right image) (magnification 40×).

FIG. 7 is a Western blot showing the detection of pimonidazole adducts in protein preparation from biopsy samples of tumors in animals given 60 mg/kg Pimonidazole 2 hours before tissue harvest. Days refer to the number of days after the tumor was implanted and tissue was harvested.

FIG. 8 is a Western blot showing the detection of pimonidazole adducts in lysates of exosomes isolated from blood in tumor bearing or non-tumor bearing mice given 60 mg/kg Pimonidazole 2 hours before blood collection. Days refer to the number of days after the tumor was implanted and blood samples were harvested for exosome isolation.

FIG. 9A presents the densitometric analysis of the detected pimonidazole adducts in each lane of the western blot image for lysates from the tumors depicted in FIG. 7. This analysis illustrates the relationship of pimonidazole adducts formed in tumor tissue after injection of pimonidazole compared to tumor tissue in mice without pimonidazole injection.

FIG. 9B presents the densitometric analysis of the detected pimonidazole adducts in each lane of the western blot image for the circulating exosomes isolated from the blood of tumor-bearing and non-tumor-bearing mice as depicted in FIG. 8. This analysis illustrates the relationship of pimonidazole adducts formed in circulating exosomes in tumor-bearing mice with and without pimonidazole injection and non-tumor-bearing mice with and without pimonidazole injection.

FIG. 10A is a chart showing the results from a flow cytometry for CD81+/Pimo+ exosomes only isolated from cells cultured in normoxic or hypoxic conditions. Bead assisted detection using commercial kit.

FIG. 10B is a chart showing the results from a flow cytometry for CD81+/Pimo+ in all vesicles isolated from hypoxic or oxic tumor cell cultures. Bead assisted detection using commercial kit.

FIG. 11 is a chart showing fluorescent plate reader results for immunocytochemical detection of pimonidazole adducts in isolated exosome samples.

### DETAILED DESCRIPTION OF THE INVENTION

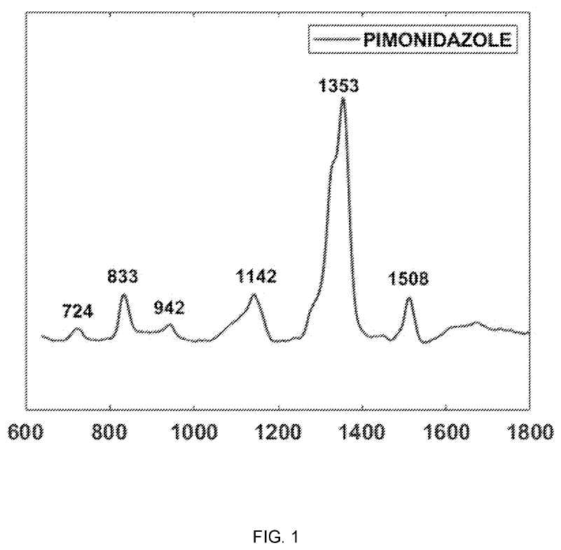

With reference to FIGS. 1-11, the preferred embodiments of the present invention may be described. The present invention is directed to methods for detecting hypoxia in a patient.

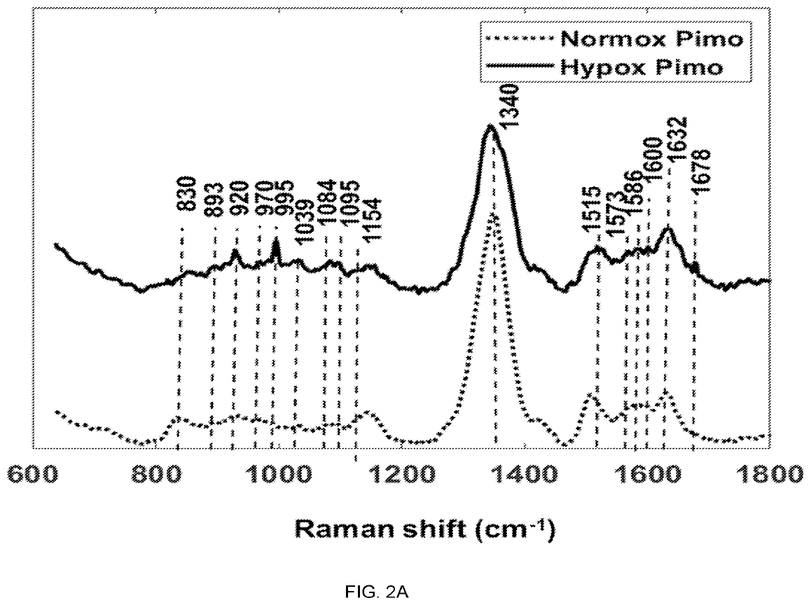

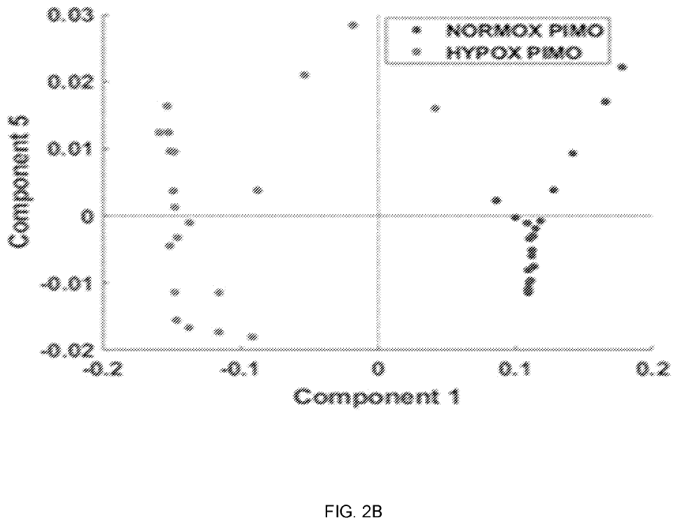

To determine if Raman spectroscopy could be used to detect pimonidazole labelling in hypoxic exosomes, the inventors first determined the Raman spectral signature of pimonidazole in solution. As shown in FIG. 1, pimonidazole generated a strong Raman signal with distinct peaks. Further, the inventors isolated exosomes from 4T1 murine breast cancer cells that were labeled with pimonidazole and either exposed to normoxic or hypoxic conditions, as shown in FIGS. 2A and 2B. FIG. 2A illustrates representative spectra from the two treatment conditions and shows key differences in spectral features between 830 and 1154 cm⁻¹. Using Principal Component Analysis (PCA), the inventors were able to clearly differentiate between measurements performed on normoxic and hypoxic exosomes (each dot in FIG. 2B represents a data point). PCA is an unsupervised algorithm used for dimensionality reduction of large datasets in machine learning. The inventors used PC component 1 and 5 (PC1 & PC5) to plot the data. PCs are numbered according to their contribution of variations between the datasets. These preliminary results demonstrate the potential of Raman spectroscopy to identify the subtle changes associated with pimonidazole labeling in hypoxic exosomes.

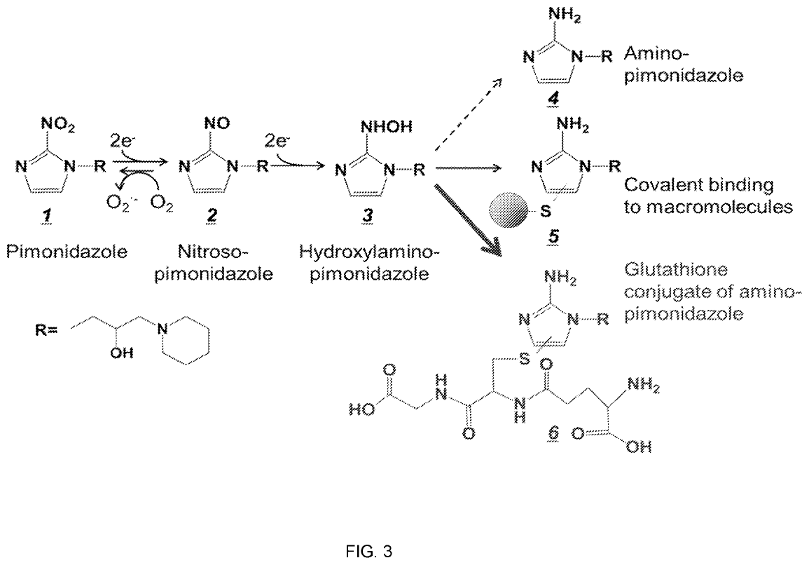

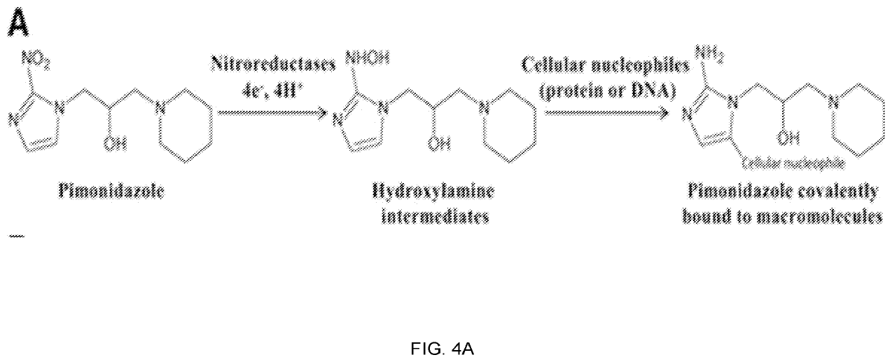

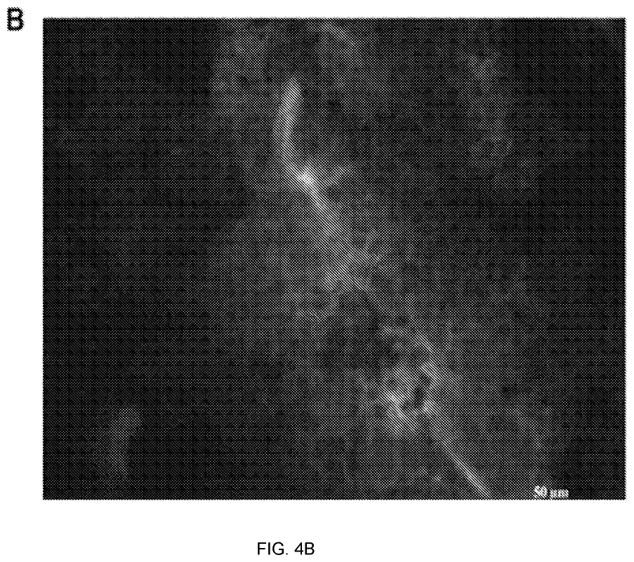

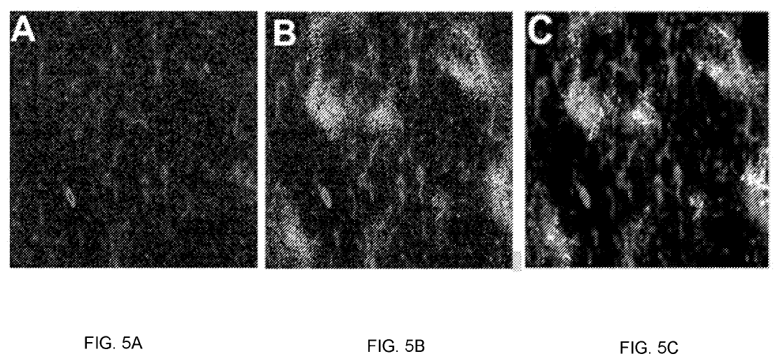

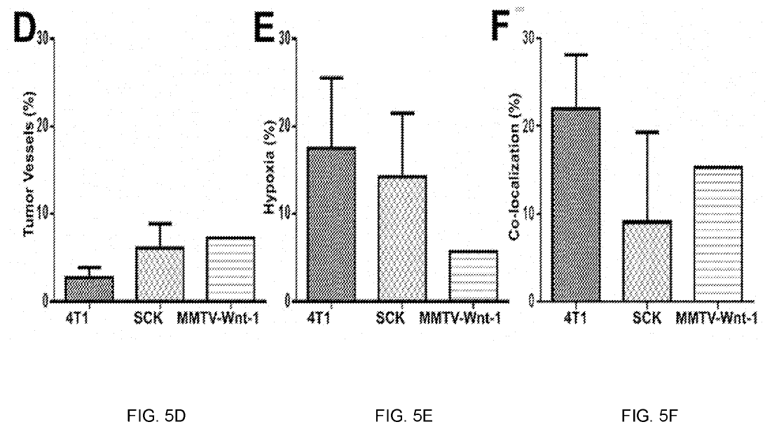

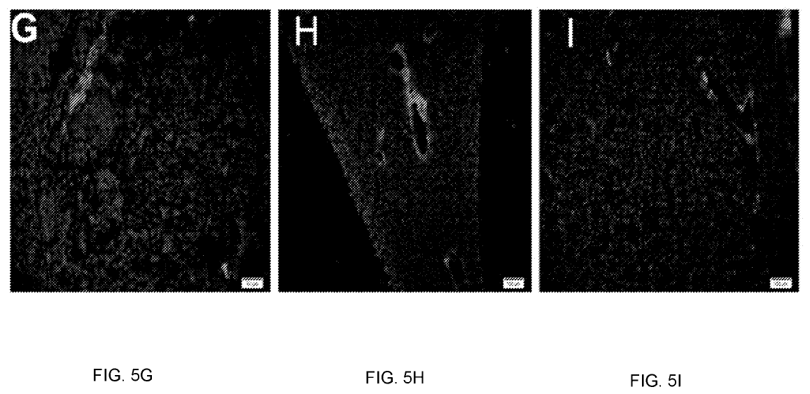

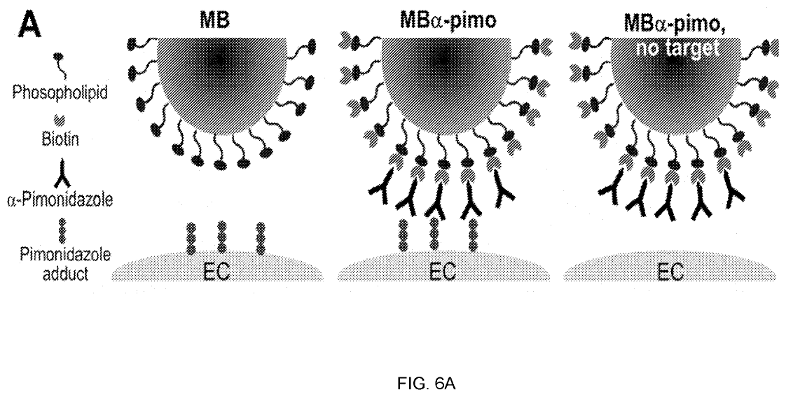

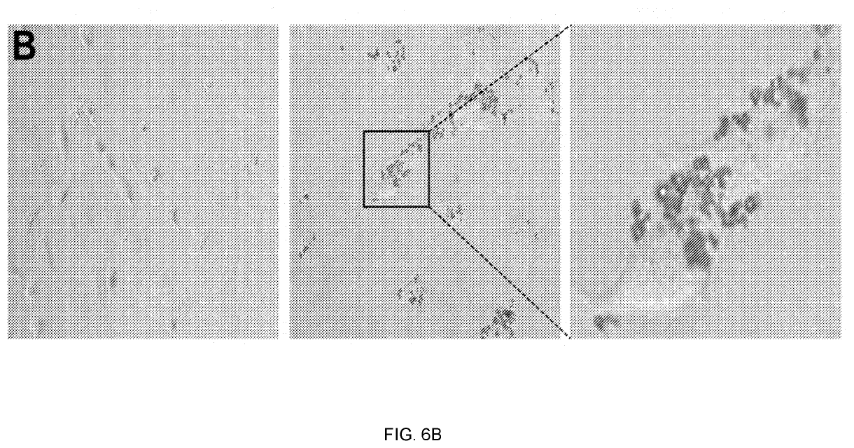

FIGS. 3-4B illustrate the reductive chemistry that occurs when nitroimidazoles bind to macromolecules in the cell in amounts inversely proportional to the amount of oxygen present. Anti-pimonidazole antibody binding with conjugated liposomes and the immunohistochemical demonstration of severe hypoxia in a tumor tissue but not in normal tissues is depicted in FIGS. 5A-6B.

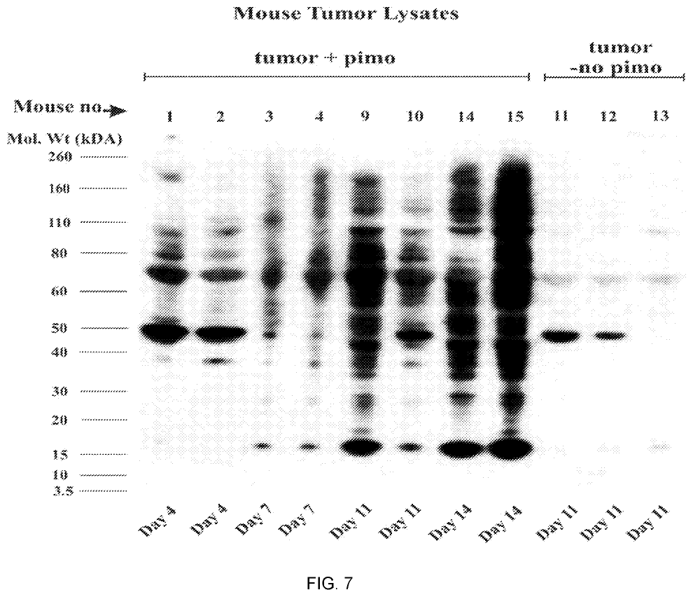

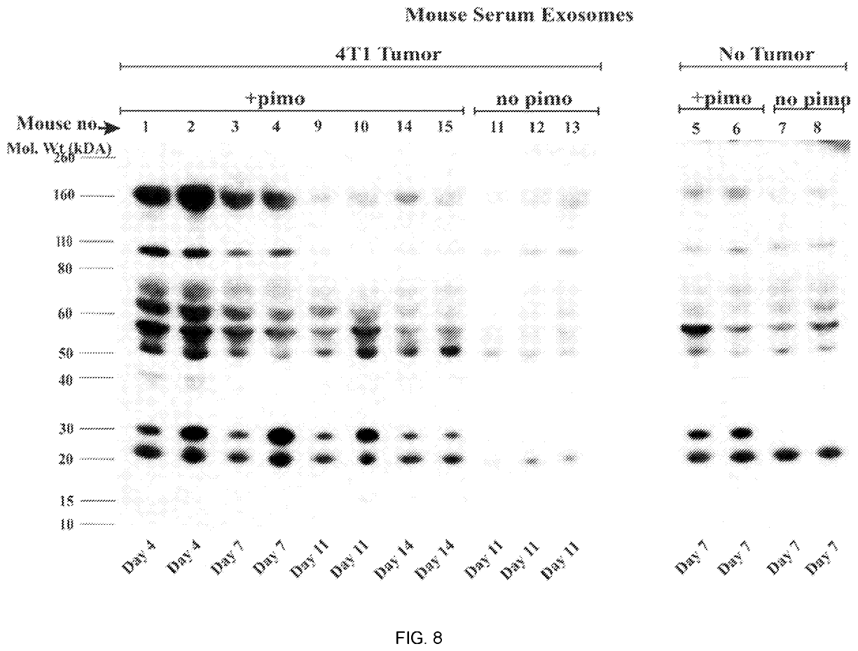

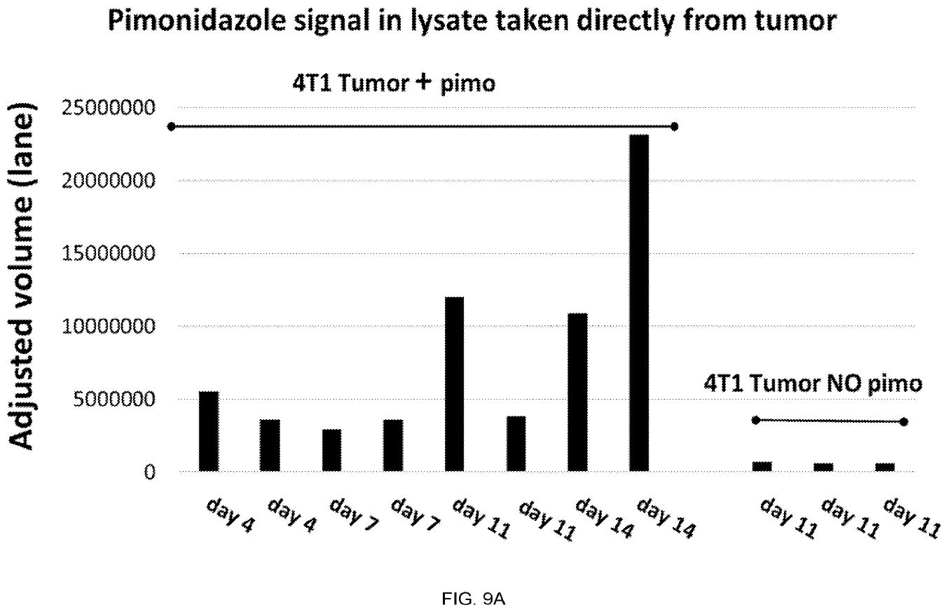

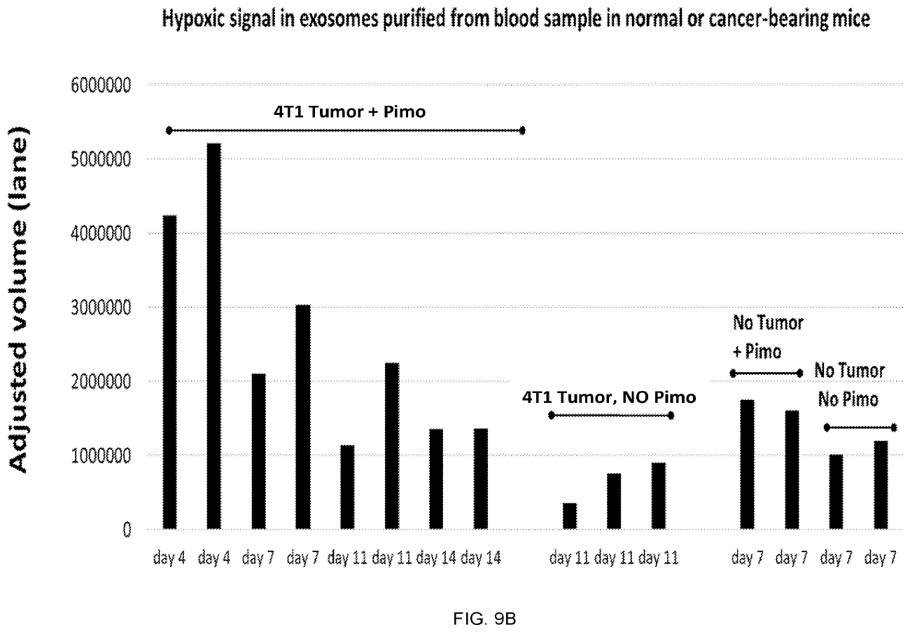

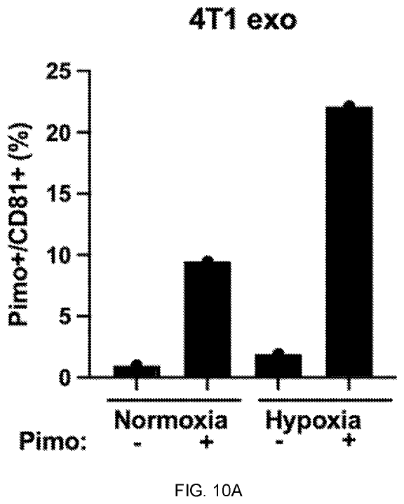

FIGS. 7-8 show the western blot results from tumor lysate or exosomes isolated from blood circulation in tumor bearing animals that were injected with pimonidazole 2 h before sacrifice at different days after the tumor cells were inoculated in the rear limb. FIG. 7 shows anti-pimonidazole antibody bound to a large fraction of the proteins present in the tumor, which suggests that there was a large degree of hypoxia in those tumors. Distinctly different, but still reflective of the presence of a hypoxic tumor mass, are the results shown in FIG. 8 where the exosome population in the circulation was isolated, and run on the gel. Anti-pimonidazole antibody clearly picked up a marked protein pattern of around 8 or 9 protein bands. A noticeable increase in binding comes from the animals that were bearing tumors and were injected with pimonidazole compared to non-tumor bearing animals or tumor bearing animals that were not injected with pimonidazole. The inventors performed densitometry analysis on each lane of the blots and found that the amount of signal in tumor-bearing mouse samples of exosomes was up to 4 fold the average result in non-tumor bearing animals. These results are illustrated in FIGS. 9A and 9B. By comparing to ‘normal’ ranges in patients, this protein binding profile can assess the total binding of anti-pimonidazole antibodies in a blood sample to predict how much hypoxia is present in the patient's body at any given timepoint, which could be linked to cancer presence and/or progression. In the same way, the presence of ischemic tissue after trauma or surgery could be assessed with this method as a semi-noninvasive readout. In the inventors' work, they have demonstrated an elevated signal from nitroimidazole labeled proteins in samples of exosomes isolated from cell culture of hypoxic cells and blood samples from animals bearing tumors using western blotting, flow cytometry and fluorescent plate reader approaches as shown in FIGS. 7-11. These are standard assays that can be run in any clinical laboratory and can be systematically validated in studies of patients with normal health status or a variety of disease or other conditions.

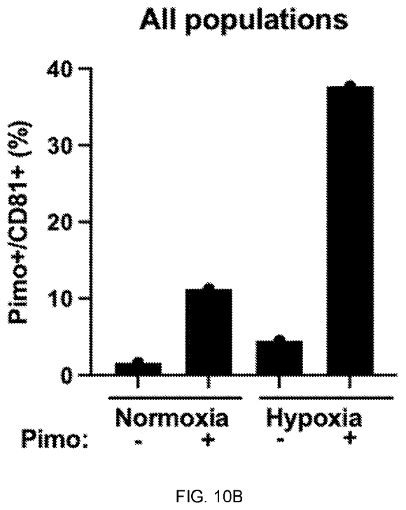

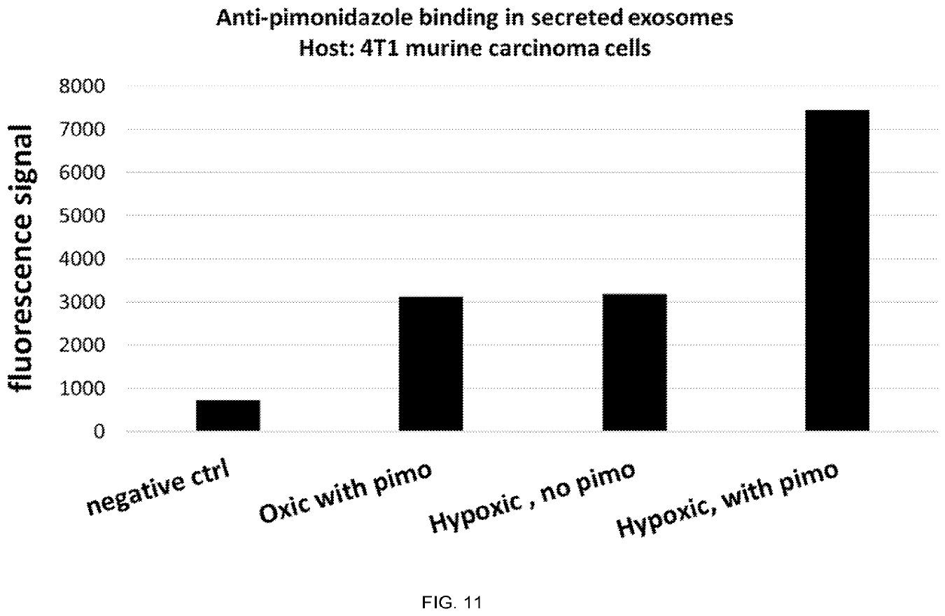

FIGS. 10A-10B are charts showing results from a flow cytometry-based bead assay designed to allow conventional flow cytometers to assess the presence of various markers on exosome sized particles. This is accomplished by using beads of known size which are bound by exosomes if they have the marker (antibody target) in question. It is clear that exosomes derived from hypoxic cells have an augmented amount of pimonidazole adducts present. The inventors also tested for the general immunoreactivity of hypoxic-cell derived exosomes using a standard plate reader and a commercially available fluorescently conjugated anti-pimonidazole antibody. Briefly, isolated exosomes from cell culture medium from mouse serum samples were, suspended in 1% bovine serum albumin, and incubated with a fluorescently tagged anti-pimonidazole antibody. After a period of 2 hours or overnight, the exosomes were washed twice, resuspended in PBS and the fluorescent signal was determined using a Cytation 5 multi-mode plate reader. As shown in FIG. 11, the signal in hypoxic-cell derived exosomes is markedly above the oxic-derived exosomes, which again suggests that a clinical diagnostic test using this approach leads to the sensitive monitoring of tumor or damaged/diseased tissue biology with only a blood sample via the amount of anti-nitroimidazole antibody binding in the sample.

The methods used for the above approaches to identify pimonidazole adducts in cells or exosomes from cell culture, tumor tissue and blood samples are performed as follows:

Mouse inoculation: To inoculate the animal with 200,000 tumor cells subcutaneously, cells were washed with serum free medium and injected subcutaneously in the right rear limb in 100 ul volume of serum-free medium. At specific timepoints (4, 7, 11 and 14 days after tumor cell inoculation), 60 mg/kg of pimonidazole was injected i.p. and after 2 h blood was collected by retro-orbital bleeds and the animals were euthanized and tumor tissue harvested for further analysis.

Tumor removal and lysate preparation: Tumors were removed and frozen immediately to prevent re-oxygenation, then pieces of tumor were lysed in ice-cold RIPA buffer with freshly added protease inhibitors cocktail and subjected to brief sonication. The homogenized lysates were then centrifuged at 12,000 rpm to remove the debris. The supernatant containing the proteins was transferred to new tubes and BCA protein assay was performed to estimate the amounts of protein. Samples were aliquoted and stored at −80 C (RIPA buffer: 25 mM Tris-HCl pH 7.5 , 150 mM NaCl, 1% NP-40, 1 mM EDTA pH 8).

Isolating exosome from cell culture and Flow Cytometry analysis of isolated exosomes utilizing bead-assisted assay (FIGS. 10A-10B): 4T1 murine breast cancer cells were cultured in DMEM medium containing 10% Fetal Bovine Serum (FBS) at 37° C. in a humidified incubator with 5% CO2 and sub-cultured twice a week. For isolation of exosomes from 4T1 cultures, 1 million cells were plated in 10 cm plates and allowed to adhere overnight in the incubator. Cells were then washed with PBS, and the medium was changed to a medium containing 5% exosome-depleted FBS. Plates were grouped into four sets of five plates each. 100 μM pimonidazole was added to two sets, half of plates were incubated in a regular air incubator, and the other half in an incubator containing 0.5% Oxygen. After 24 hours, the medium was collected and subjected to sequential centrifugation at 1200, 3000, 10000, and 35000 rpm for 5, 15, 45 minutes, and 4 hours respectively. The final pellet was washed with PBS and centrifuged at 35000 rpm for 2 hours before being resuspended in 100 μl of PBS. The exosome amount was estimated using a BCA protein assay and the exosomes size was confirmed using a Nanoparticle Tracking Analyzer (NTA Instrument). The PS Capture Exosome Flow Cytometry Kit (FUJIFILM Wako Pure Chemical Corporation) was utilized to assess the binding of anti-pimonidazole antibody (FITC labeled, 494/520 nm) as well as the CD81 (PE-Cy7 labeled, 496/785 nm), an exosomal marker, to the exosomes derived from cell cultures containing pimonidazole which were subjected to hypoxic (0.5% oxygen) conditions. Following the manufacture protocols, compensation beads were used to optimize fluorescence compensation settings and correct for spectral overlap between fluorophores. For the single stain controls, for the pimonidazole antibody which was from mouse, mouse comp beads were used. For the CD81, which was from hamster, rat/hamster compensation beads were used.

Western blotting (FIGS. 7-8): Equal amounts of tumor or exosome proteins were loaded per lane of a 4-15% Mini-PROTEAN TGX SDS-polyacrylamide gel under denaturing and reducing conditions, followed by electrophoresis to separate the proteins. The separated proteins were then transferred to a polyvinylidene fluoride (PVDF) membrane. Nonspecific binding sites on the membrane were blocked with 5% non-fat powdered milk in TBS-T (0.5% Tween-20), after which the membrane was probed with a mouse FITC conjugated anti-pimonidazole primary antibody overnight on a shaker in cold condition. Subsequently, the membrane was washed three times with TBS-T (0.5% Tween-20) and incubated with an anti-FITC secondary antibody conjugated to horse radish peroxidase (HRP) for two hours to bind to the primary antibody. The membrane was washed and bands corresponding to the target protein were then detected utilizing enhanced chemiluminescence reagents and a Bio-Rad imaging apparatus. Densitometry analysis of the detected bands was performed using Image lab software to estimate the amount of pimonidazole-protein adduct formation in each sample.

Exosome isolation from mouse blood: Blood was collected via retro-orbital bleeds into gold cap tubes and centrifuged at 4000 G for 10 minutes to separate serum. Exosomes were isolated from serum utilizing the commercial kit, Total Exosome Isolation from Serum Reagent from Invitrogen, following the manufacturer's instructions. Briefly, the serum was centrifuged for 30 minutes at 2000 g in cold conditions, and the clarified serum was then transferred to new tubes. Next, 0.2 volumes of the reagent were added and immediately vortexed. The mixture was left on ice for 10 minutes before being spun at 10,000 G force at room temperature. Following removal of the supernatant, the pellet containing exosomes was resuspended in 100 μl PBS. For further purification, the exosomes underwent sequential centrifugation at 10,000 rpm and 35,000 rpm in cold conditions for 15 minutes and 2 hours, respectively. The purified exosome pellet was then resuspended in 50 μl PBS, and a BCA protein assay was employed to estimate the protein amount.

Labeling exosomes with FITC-conjugated anti-pimonidazole antibody and quantifying the labeled exosomes'(FIG. 11): 10-40 μg of exosomes were utilized for antibody labeling in a 100 μl total volume containing 1% BSA as a blocking reagent. Following a one-hour blocking step at room temperature with periodic shaking, 10 μl of 1:10 diluted FITC-labeled anti-pimonidazole antibody (hypoxiaprobe-1 antibody, Mab1-FITC) was added to each tube, followed by overnight incubation with gentle shaking at cold temperatures. Subsequently, ultracentrifugation was conducted at 35,000 rpm for 2 hours, followed by removal of the supernatant, washing with 1 ml PBS, and spinning again for 2 hours at 35,000 rpm. After discarding the supernatant, the pellet was resuspended in 50 μl PBS. Fluorescence readings were obtained using a Cytation 5 plate reader at excitation/emission wavelengths of 485/528 nm (+/−20 nm).

It should be understood that there are a number of other approaches that could be tested and have even greater sensitivity than the assays and results presented here, such as radioisotope labeled antibodies or Raman-enhanced tagged antibodies, yet all the sample labeling and collection strategies would be the same as described here.

In one embodiment, the present method is directed to the use of shed or secreted vesicles in the blood stream from a patient first treated with a hypoxia-specific probe to detect hypoxia in the patient. In another embodiment, the present invention is directed to a method of detecting hypoxia in a patient by detecting vesicles secreted by cells in hypoxic tissue environments that are labeled as a result of the formation of chemical adducts (i.e., nitroimidazole-protein bonds). The detection array of methods is comprised of Raman spectroscopy or a variety of standard cell biology/antibody or other probe detections of the nitroimidazole-protein bonds to allow development of clinical laboratory based assessments comparing each patient's result to statistically validated values/ranges for each assay obtained from healthy individuals.

In application, the present method preferably includes the following: (1) injecting or administering to a patient an effective dose of a nitroimidazole usually in the range of 1-75 mg/kg. The nitroimidazole may be pimonidazole, etanidazole, or misonidazole-all of which are similarly reactive; (2) after at least 1 hour and up to 72 hours after injection or administration, a standard blood sample and serum preparation is made; (3) the serum is used to isolate the vesicle fraction (containing exosomes and other vesicles) according to the methods described above; (4) the isolated fraction is subjected to either Raman spectroscopy or a battery of cell biology assays that would be well-understood by a person of ordinary skill in the art to detect nitroimidazole binding in the sample as an indicator of the amount of hypoxic cells in the patient and compared to a range of values found in normal/healthy individuals. The cell biology assays may be any of the following: (1) Western blotting for nitroimidazole containing proteins (as described in FIGS. 7-8 and associated methods described above); (2) flow cytometry using bead assisted sizing for nitroimidazole containing proteins; or (3) plate reader assays, similar to or including ELISA, where a sample of isolated vesicles is exposed to a fluorescently conjugated antibody against nitroimidazole adducts. The spectroscopic or fluorescent/densitometry/flow cytometry results are compared to an established “normal individual range” and used as a prognostic or predictive indicator of diseased tissue presence, ischemic after surgery or transplant or progression of solid tumor growth before, during or after any cancer therapy.

The present invention has been described with reference to certain preferred and alternative embodiments that are intended to be exemplary only and not limiting to the full scope of the present invention.

We claim:

1. A method for detecting hypoxia in a patient, comprising the steps of:
(a) administering to said patient an effective amount of a nitroimidazole;
(b) drawing blood from said patient and separating a serum of said blood;
(c) isolating an extracellular vesicle fraction from said serum; and
(d) conducting a test for detecting said nitroimidazole in said extracellular fraction, wherein a test result from said test indicates an amount of hypoxic cells in said patient.

2. The method of claim 1, further comprising the step of comparing said test result to a normal test result to determine the presence or absence of a medical condition.

3. The method of claim 1, wherein said step (b) is performed at least one hour and no later than 72 hours after said step (a).

4. The method of claim 1, wherein said effective amount of said nitroimidazole is 1 mg/kg-75 mg/kg.

5. The method of claim 1, wherein said nitroimidazole is a pimonidazole, an etanidazole, or a misonidazole.

6. The method of claim 1, wherein said test comprises a Western blot.

7. The method of claim 1, wherein said test comprises a flow cytometry.

8. The method of claim 1, wherein said test comprises a plate reader assay.

9. The method of claim 1, wherein said test comprises a Raman spectroscopy.

10. The method of claim 8, wherein said plate reader assay comprises fluorescently-conjugated antibodies against said nitroimidazole.

11. The method of claim 2, wherein said medical condition comprises a diseased tissue.

12. The method of claim 2, wherein said medical condition comprises a cancer.

13. The method of claim 2, wherein said medical condition comprises ischemia after an organ or tissue transplant.
